(19) United States
(12) Patent Application Publication
(10) Pub. No.: US 2021/0322497 A1
(43) Pub. Date: Oct. 21, 2021

(54) Method of Fermenting Soybeans or other Oil Crops by Edible and Medicinal Fungi

(71) Applicant: Jiangnan University, Wuxi (CN)

(72) Inventors: Zhongyang DING, Wuxi (CN); Yuanfa LIU, Wuxi (CN); Yunzhi JIANG, Wuxi (CN); Mengmeng XU, Wuxi (CN); Liting ZHAO, Wuxi (CN); Chen CAO, Wuxi (CN); Qiong WANG, Wuxi (CN); Zhenghua GU, Wuxi (CN); Guiyang SHI, Wuxi (CN)

(21) Appl. No.: 17/352,259

(22) Filed: Jun. 19, 2021

Related U.S. Application Data
(63) Continuation of application No. PCT/CN2018/123845, filed on Dec. 26, 2018.

(30) Foreign Application Priority Data
Dec. 20, 2018 (CN) 2018115618575

(51) Int. Cl. A61K 36/068; A61K 36/48; A61K 36/07; A61K 36/31; A23L 33/115; A23L 11/50; A23L 25/00; A23L 11/00; A23K 10/12

(52) U.S. Cl. CPC A61K 36/068; A61K 36/48; A61K 36/07; A61K 36/31; A23L 33/115; A23V 2002/00; A23L 25/40; A23L 11/05; A23L 11/07; A23K 10/12; A61K 2236/19; A23L 11/50

(57) ABSTRACT

The disclosure discloses a method of fermenting soybeans or other oil crops by edible and medicinal fungi, and belongs to the technical field of fermentation. The method involves solid-state fermentation by inoculating edible and medicinal fungi such as i Ganoderma lucidum, Tremella aurantialba, Hericium erinaceus and Cordyceps sinensis that can produce bioactive substances such as active polysaccharides, flavonoids and triterpenoids in a fermentation substrate containing oily crops such as extruded soybeans, peanuts and rapeseeds, thereby obtaining oil and oil crop meal rich in the bioactive substances. In the oil obtained by the method, the content of flavonoids is as high as 0.302 mg/g, and the content of triterpenoids is as high as 65.663 mg/g. In the oil crop meal, the content of active polysaccharides is as high as 60.651 mg/g, the content of flavonoids is as high as 1.599 mg/g, and the content of triterpenoids is as high as 14.225 mg/g.

### TECHNICAL FIELD

The disclosure relates to a method of fermenting soybeans or other oil crops by edible and medicinal fungi, and belongs to the technical field of fermentation.

### BACKGROUND

Soybeans are an important grain and oil crop in the world, and about 85% of soybean production in the world is used for oil extraction. Therefore, soybeans are the most important sources of vegetable oil and protein feed in the world. An existing soybean processing technology for preparing soybean oil and soybean meal mainly includes first extruding soybeans, extracting oil to obtain the soybean oil, and then drying the residue to obtain the soybean meal. The extracted soybean oil is excellent edible oil with very high nutritional value, and is one of the most common edible oil in people's daily life. The processed soybean meal is a by-product after the soybean oil is produced. Not only is the output of the soybean meal huge, but also the protein content of the soybean meal is as high as 45% or above. The amino acid composition is relatively reasonable, so the soybean meal is a high-quality vegetable protein source for culture feed.

In terms of soybean oil, at present, with the continuous improvement of the quality of life, consumers have higher and higher requirements on the quality of edible oils. In order to gain the favor of consumers, researchers and manufacturers of edible oils have been trying to add natural functional substances (such as carotenoid, squalene and flavone) into soybean oil to endow the soybean oil with certain bioactivities, so that obtained products, functional oils, not only have higher market competitiveness, but also have improved additional value. However, the acquisition of those natural functional substances requires a process of production, extraction and preparation; thus, such oil products have a defect of long technological process in production or natural functional substance products need to be purchased for addition at a high cost.

In terms of soybean meal, researchers and manufacturers of feed often carry out microbial fermentation of bean meal to further improve the quality of the bean meal. However, the acquisition of the fermented bean meal needs a plurality of links of water adding and material mixing, raw material sterilizing, inoculating and fermenting and drying; thus, although the quality of the bean meal is improved, the production cost is greatly increased, which is an important problem to be solved for the feed and culture industry that needs to strictly control cost.

### SUMMARY

The present disclosure provides a method of fermenting extruded soybeans or other oil crops by edible and medicinal fungi. The method involves solid-state fermentation by inoculating edible and medicinal fungi such as Ganoderma lucidum, Tremella aurantialba, Hericium erinaceus and Cordyceps sinensis that can produce bioactive substances such as active polysaccharides, flavonoids and triterpenoids in a fermentation substrate containing oil crops such as extruded soybeans, peanuts and rapeseeds, thereby obtaining oil rich in flavonoids and triterpenoids, and oil crop meal rich in active polysaccharides, flavonoids and triterpenoids. In the oil obtained by the method, the content of flavonoids is as high as 0.302 mg/g, and the content of triterpenoids is as high as 65.663 mg/g. In the oil crop meal, the content of active polysaccharides is as high as 60.651 mg/g, the content of flavonoids is as high as 1.599 mg/g, and the content of triterpenoids is as high as 14.225 mg/g. The above active substances have bioactivity in resisting oxidation, delaying aging, enhancing immunity, preventing cancer, etc. By the method, only one step of fermentation and one step of extraction of oil are required, and oil and oil crop meal rich in bioactive ingredients can be simultaneously obtained without extra addition, which greatly reduces the production cost of functional oil and fermented oil crop meal.

The disclosure provides a method of fermenting soybeans or other oil crops by edible and medicinal fungi. The method includes the following steps: first, inoculating edible and medicinal fungi into a fermentation medium for performing solid-state fermentation to obtain a fermented product after solid-state fermentation, and then pressing the fermented product to obtain oil and oil crop meal.

The edible and medicinal fungi are edible and medicinal fungi capable of producing bioactive substances.

The bioactive substances include one or more of active polysaccharides, flavonoids and triterpenoids.

Components of the fermentation medium include oil crop powder and water.

The oil crop powder includes one or more of soybean extruded powder, soybean powder, peanut powder, rapeseed powder, castor powder and sesame powder.

In one embodiment of the disclosure, the fermentation medium includes the oil crop powder accounting for 40-50% of the total mass of the fermentation medium and the water accounting for 50-60% of the total mass of the fermentation medium.

In one embodiment of the disclosure, the fermentation medium includes the oil crop powder accounting for 40% of the total mass of the fermentation medium and the water accounting for 60% of the total mass of the fermentation medium.

In one embodiment of the disclosure, the fermentation medium includes the soybean extruded powder and water.

In one embodiment of the disclosure, the edible and medicinal fungi include one or more of Ganoderma lucidum, Tremella aurantialba, Hericium erinaceus and Cordyceps sinensis.

In one embodiment of the disclosure, the edible and medicinal fungus is Ganoderma lucidum.

In one embodiment of the disclosure, conditions for the solid state fermentation include the temperature of 25-30° C. and the time of 10-20 d.

In one embodiment of the disclosure, the conditions for the solid state fermentation include the temperature of 30° C. and the time of 10 d.

In one embodiment of the disclosure, in inoculating the edible and medicinal fungi into the fermentation medium for performing solid-state fermentation, an edible and medicinal fungal seed liquid is inoculated into the fermentation medium for performing solid-state fermentation; the inoculation amount of the edible and medicinal fungal seed liquid in the fermentation medium is that the volume of the edible and medicinal fungal seed liquid accounts for 5-20% of the mass of the oil crop powder; and the edible and medicinal fungal seed liquid is obtained by inoculating an edible and medicinal fungus into a seed culture medium. The inoculation amount of 5-20% means that 5-20 mL of edible and medicinal fungal seed liquid is inoculated per 100 g of oil crop powder.

The disclosure provides oil and oil crop meal prepared by the above method.

The disclosure provides a product containing the above oil and/or oil crop meal.

In one embodiment of the disclosure, the product is a feed, a food, a drug or a healthcare product.

The oil prepared by the method of the disclosure is rich in bioactive substances such as flavonoids and triterpenoids; the content of flavonoids is as high as 0.302 mg/g, and the content of triterpenoids is as high as 65.663 mg/g. Therefore, the oil prepared by the method of the disclosure has anti-cancer, anti-oxidation and other effects, and has great application prospects in the fields of food, medicine and health care products.

The existing functional oil needs to be prepared by preparing and extracting nutrients at first, and then blending oil with various nutrients that can regulate human health, so the process is complicated and the cost is high. In the disclosure, edible and medicinal fungi capable of producing bioactive ingredients are directly inoculated into a fermentation medium formed by mixing oil crop powder rich in oil, such as puffed soybeans, peanuts and rapeseeds, and water for performing solid-state fermentation, thereby obtaining functional oil rich in flavonoids, triterpenoids and other bioactive substances; the operation is simple and cost is low.

The oil crop meal such as soybean meal, peanut meal and rapeseed meal prepared by the method of the disclosure is rich in bioactive substances such as active polysaccharides, flavonoids and triterpenoids; the content of active polysaccharides is as high as 60.651 mg/g, the content of flavonoids is as high as 1.599 mg/g, and the content of triterpenoids is as high as 14.225 mg/g. Therefore, the oil crop meal prepared by the method of the disclosure has anti-cancer, anti-oxidation and other effects, and has great application prospects in preparation of high-quality feed.

Existing feed manufacturers often sterilize the oil crop meal after oil extraction and then ferment the oil crop meal with various fungi to make fermented soybean meal feed, which undoubtedly adds extra steps such as sterilization and fermentation to the processing of feed, and greatly increases the cost of feed. In the disclosure, edible and medicinal fungi capable of producing bioactive ingredients are directly inoculated into the fermentation medium formed by mixing oil crop powder rich in oil, such as extruded soybeans, peanuts and rapeseeds, and water for performing solid-state fermentation. The oil crop meal obtained after oil extraction has realized the original purpose and significance of the fermentation of oil crop meal, and at the same time, the operation procedures are reduced, which is conducive to greatly reducing the cost.

In order to improve the survival rate of animals being raised, feed manufacturers often add a large amount of antibiotics into feed, which will undoubtedly cause a large amount of antibiotics to remain in the body of the animals being raised, and cause hidden dangers to the hygiene and safety of health food. In the disclosure, edible and medicinal fungi capable of producing bioactive ingredients are directly inoculated into the fermentation medium formed by mixing oil crop powder rich in oil, such as extruded soybeans, peanuts and rapeseeds, and water for performing solid-state fermentation, thereby obtaining oil crop meal which is rich in active substances such as polysaccharides, triterpenes and flavonoids, and has the effects of resisting oxidation, delaying aging, enhancing immunity, preventing cancer, etc. The oil crop meal has great potential to completely or partially replace the antibiotics in the feed, and has the effect of improving the survival rate of the animals being raised.

### BRIEF DESCRIPTION OF FIGURES

FIG. 1 shows functional oil prepared by solid-state fermentation of puffed soybeans with different edible and medicinal fungi.

### DETAILED DESCRIPTION

The disclosure is further described in conjunction with Examples as follows.

Ganoderma lucidum 5.26, Tremella aurantialba, Hericium erinaceus and Cordyceps sinensis involved in the following examples are all purchased from the China General Microbiological Culture Collection Center, with the numbers of CGMCC No. 5.26, CGMCC No. 5.506, CGMCC No. 5.111 and CGMCC No. 3.15498 respectively. Soybean extruded powder involved in the following examples are from Shandong Bohai Oil Industry Co., Ltd., and peanuts and rapeseed are purchased from Wuxi Farmers Market (the above strains of Ganoderma lucidum CGMCC No. 5.26, Tremella aurantialba CGMCC No. 5.506, Hericium erinaceus CGMCC No. 5.111, and Cordyceps sinensis CGMCC No. 3.15498 are all available for purchase and do not need to be deposited for patent procedures).

The Culture Media Involved in the Following Examples are as Follows:

Seed culture medium for edible and medicinal fungi (m/v): peptone 1%, yeast powder 0.5%, glucose 2%, potassium dihydrogen phosphate 0.1%, magnesium sulfate heptahydrate 0.1%, and VB 0.01%.

Detection Methods Involved in Following Examples are as Follows:

Analysis of Functional Oil and Oil Crop Meal:

1. Extraction

A product after solid-state fermentation is dried at 50° C. until the water content reaches 5-10%; the dried fermented product and n-hexane are mixed at a mass ratio of 1:30, soaked and stirred for 24 h; suction filtration is performed and a filtrate is taken; and the filtrate is subjected to rotary evaporation at 50° C. by a rotary evaporator to obtain the functional oil and oil crop meal.

2. Measurement of Type and Content of Fatty Acid in Functional Oil

50 mg of the functional oil is added to 2 mL of a 0.5 mol/L NaOH—CH₃OH solution, and saponified in a water bath at 65° C. for 30 min. After being cooled to room temperature, the functional oil is added to 2 mL of a 14% BF₃—CH₃OH solution, and saponified in a water bath at 65° C. for 30 min. After being cooled to room temperature, the functional oil is added to 5 mL of n-hexane and shaken for 3-4 min to extract fatty acid methyl ester. The fatty acid methyl ester is added to a small amount of anhydrous Na₂SO₄ for dehydration. Centrifugation is performed at 10000 r/min for 5 min. The upper organic phase is filtered with a 0.22 μm organic membrane for later use. 0.2 mg/mL methyl nonadecanate solution (using n-hexane as a solvent) is added as an internal standard to the solution after methyl esterification and filtration at a volume ratio of 1:1. The content of fatty acid in the extract is measured by GC-MS.

3. Extraction and Content Measurement of Bioactive Substances in Functional Oil

(1) Extraction and Measurement of Flavonoids in Functional Oil

200 mg of the functional oil is accurately weighed, added to 4 mL of 70% ethanol, and extracted in a water bath at 80° C. for 2 h. 1 mL of a flavonoid extract diluted by an appropriate multiple is added into a 10 mL colorimetric tube, 0.2 mL of a 5% NaNO₂ solution is added in turn and shaken well, and the mixed solution is allowed to stand for 5 min. 0.2 mL of a 10% Al(NO₃)₃ solution is added and the mixed solution is allowed to stand for 6 min. 7 mL of 1 mol/L NaOH is added and shaken well. The mixed solution is diluted to volume with 50% ethanol and shaken well. The solution will be brown-red, and is allowed to stand for 15 min. Distilled water is used as a blank control, and the OD value measured at the absorbance wavelength of 510 nm is substituted into a standard curve to calculate the content of flavonoids.

Making of standard curve: 12.5 mg of standard rutin by dry and constant weight at 120° C. is accurately weighed, added to 50% ethanol, dissolved and diluted to 50 mL, thereby preparing a standard with the concentration of 0.25 mg/mL. 0.0, 0.5, 1.0, 1.5, 2.0 and 2.5 mL of standard solutions are accurately pipetted and added into a 10 mL colorimetric tube, and 0.2 mL of a 5% NaNO₂ solution is added in turn. The mixed solutions are shaken well and allowed to stand for 5 min. 0.2 mL of a 10% Al(NO₃)₃ solution is added and the mixed solution is allowed to stand for 6 min. 7 mL of 1 mol/L NaOH is added and shaken well. The mixed solution is diluted to volume with 50% ethanol and shaken well. The solution will be brown-red, and is allowed to stand for 15 min. No. 0 is used as a blank control, the OD value at the absorbance wavelength of 510 nm is measured, and a curve with absorbance A as the abscissa and concentration as the ordinate is drawn.

(2) Extraction and Measurement of Triterpenoids in Functional Oil

0.2 g of functional oil is precisely weighed, added into a 10 mL volumetric flask, dissolved with methanol, diluted to the volume, and extracted with ultrasound for 2 h (shaken well every 20 min). 1 mL of an extract solution is added in a 1.5 mL centrifuge tube and centrifuged at 4000 rpm for 10 min, and the supernatant is taken for later use. 0.1 mL of a sample solution is evaporated to dryness in a boiling water bath, then 0.2 mL of a 5% vanillin solution and 0.5 mL of perchloric acid are added respectively, and the mixed solution is placed in a water bath at 60° C. for 20 min. The mixed solution is transferred to an ice-water bath for cooling, and then 5.0 mL of glacial acetic acid is added and shaken well. the OD value at the absorbance wavelength of 548 nm is measured by a spectrophotometer, and substituted into a standard curve to calculate the content.

Making of standard curve: 0.10 mL, 0.20 mL, 0.40 mL, 0.60 mL, 0.80 mL, 1.00 mL and 1.20 mL of 0.1 mg/mL ursolic acid solutions are accurately pipetted and added into a 10 mL colorimetric tube with a stopper, and heated in a water bath to evaporate the solvent. 0.2 mL of a freshly prepared 5% vanillin-glacial acetic acid solution and 0.8 mL of a perchloric acid solution are added, and the mixed solution is heated in a 65° C. water bath for 15 min. The mixed solution is taken out and cooled in an ice bath, 5.0 mL of glacial acetic acid is added, and the mixed solution is shaken well, allowed to stand for 15 min, and measured at the wavelength of 548 nm. Linear regression is performed on concentration (C) with absorbance (A) to obtain a regression equation.

4. Extraction and Content Measurement of Bioactive Substances in Oil Crop Meal

(1) Extraction and Measurement of Polysaccharides in Oil Crop Meal

20 mg of oil crop meal is added to 4 mL of water, boiled in a boiling water bath for 3 h, and centrifuged at 10000 r/min for 10 min, and the supernatant is taken and diluted to volume for testing. 100 μL of supernatant solution is added to 1.9 mL of deionized water, and the control group is 2 mL of deionized water. 1 mL of a 60% phenol solution is added. 5 mL of concentrated sulfuric acid is added and shaken well. After cooling, the OD value at the absorbance wavelength of 490 nm is measured and substituted into a standard curve to calculate the content of polysaccharides.

Making of standard curve: a 0.04 g/L glucose standard solution is prepared. 0, 0.2, 0.4, 0.6, 0.8, 1.0, 1.2, 1.4, 1.6, 1.8 and 2.0 mL of standard solutions are pipetted and added into a 25 mL colorimetric tube respectively. 2.0, 1.8, 1.6, 1.4, 1.2, 1.0, 0.8, 0.6, 0.4, 0.2 and 0 mL of deionized water is added respectively. 1 mL of a 60% phenol solution is added, and finally 5 mL of concentrated sulfuric acid is added and shaken well. After cooling, the OD value is measured at the absorption wavelength of 490 nm, and the standard curve is drawn.

(2) Extraction and Measurement of Flavonoids in Oil Crop Meal

200 mg of oil crop meal is added into 4 mL of 70% ethanol, and extracted at 80° C. for 2 h. 1 mL of a flavonoid extract diluted by an appropriate multiple is added into a 10 mL colorimetric tube, 0.2 mL of a 5% NaNO₂ solution is added in turn and shaken well, and the mixed solution is allowed to stand for 5 min. 0.2 mL of a 10% Al(NO₃)₃ solution is added and the mixed solution is allowed to stand for 6 min. 7 mL of 1 mol/L NaOH is added and shaken well. The mixed solution is diluted to volume with 50% ethanol and shaken well. The solution will be brown-red, and is allowed to stand for 15 min. Distilled water is used as a blank control, and the OD value measured at the absorbance wavelength of 510 nm is substituted into a standard curve to calculate the content of flavonoids.

Making of standard curve: 12.5 mg of standard rutin by dry and constant weight at 120° C. is accurately weighed, added to 50% ethanol, dissolved and diluted to 50 mL, thereby preparing a standard with the concentration of 0.25 mg/mL. 0.0, 0.5, 1.0, 1.5, 2.0 and 2.5 mL of standard solutions are accurately pipetted and added into a 10 mL colorimetric tube, and 0.2 mL of a 5% NaNO₂ solution is added in turn. The mixed solutions are shaken well and allowed to stand for 5 min. 0.2 mL of a 10% Al(NO₃)₃ solution is added and the mixed solution is allowed to stand for 6 min. 7 mL of 1 mol/L NaOH is added and shaken well. The mixed solution is diluted to volume with 50% ethanol and shaken well. The solution will be brown-red, and is allowed to stand for 15 min. No. 0 is used as a blank control, the OD value at the absorbance wavelength of 510 nm is measured, and a curve with absorbance A as the abscissa and concentration as the ordinate is drawn.

(3) Extraction and Measurement of Triterpenoids in Oil Crop Meal

0.5 g of a sample is accurately weighed, added into a 50 mL volumetric flask, dissolved with 35 mL of ethyl acetate, and extracted with ultrasonic vibration for 30 min. The extract solution is taken out and cooled to room temperature, diluted to the volume with ethyl acetate, shaken well, allowed to stand, and filtered. A primary filtrate is discarded and a subsequent filtrate is kept as a sample extract solution for later use. The sample extract solution is diluted to an appropriate multiple and measured at the wavelength of 548 nm.

Making of standard curve: 0.10 mL, 0.20 mL, 0.40 mL, 0.60 mL, 0.80 mL, 1.00 mL and 1.20 mL of 0.1 mg/mL ursolic acid solutions are accurately pipetted and added into a 10 mL colorimetric tube with a stopper, and heated in a water bath to evaporate the solvent. 0.2 mL of a freshly prepared 5% vanillin-glacial acetic acid solution and 0.8 mL of a perchloric acid solution are added, and the mixed solution is heated in a 65° C. water bath for 15 min. The mixed solution is taken out and cooled in an ice bath, 5.0 mL of glacial acetic acid is added, and the mixed solution is shaken well, allowed to stand for 15 min, and measured at the wavelength of 548 nm. Linear regression is performed on concentration (C) with absorbance (A) to obtain a regression equation.

### Example 1: Using Soybean Extruded Powder as Raw Material

Specific steps are as follows:

(1) 80 mL of a seed culture medium was added into a 250 mL erlenmeyer flask (sterilized at 121° C. for 30 min). 1 cm² cube of an edible and medicinal fungal strain stored at 4° C. was inoculated into the sterilized liquid seed culture medium, and cultured at 30° C. and 150 r·min⁻¹ for 7 d to obtain seed liquid.

(2) 300 g of a fermentation medium was added into a 2500 mL erlenmeyer flask (sterilized at 121° C. for 30 min), and the seed liquid was inoculated into the fermentation medium according to fermentation parameters in Table 1 for performing solid-state fermentation to obtain a fermented product.

Oil extraction was performed on the obtained fermented product by analysis methods of functional oil and oil crop meal to obtain the functional oil and fermented bean meal; and the obtained functional oil and fermented bean meal were analyzed by the analysis methods of functional oil and oil crop meal. Analysis results are shown in Tables 2-4. The blank group is original soybean extruded powder without microbial fermentation.

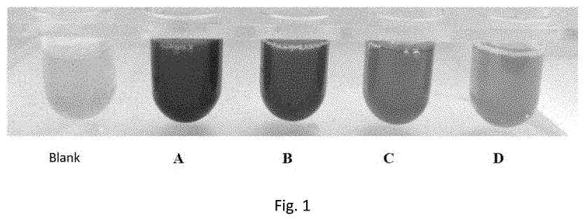

The functional oil extracted from the original soybean extruded powder and group A, group B, group C, and group D are shown in FIG. 1. The oil extracted from the original soybean extruded powder is golden yellow; the functional oil obtained in group A is dark brown; the functional oil obtained in group B is lighter than group A and is light brown; the functional oil obtained in group C is lighter than group B and is dark orange; and the functional oil obtained in group D is the lightest in color, which is light orange.

TABLE 1
Fermentation parameters

| | | | | | Fermenta- |
|---|---|---|---|---|---|
| | | | Inoculation | | tion |
| Group | | Strain | amount | Culture medium | conditions |
| Group | Group | Ganoderma | 18 mL | Soybean | 30° C., |
| A | A1 | lucidum | | extruded powder, | 10 d |
| | | 5.26 | | 120 g; water, 180 g | |
| | Group | Ganoderma | 7 mL | Soybean | 25° C., |
| | A2 | lucidum | | extruded powder, | 15 d |
| | | 5.26 | | 140 g; water, 160 g | |
| | Group | Ganoderma | 22 mL | Soybean | 28° C., |
| | A3 | lucidum | | extruded powder, | 20 d |
| | | 5.26 | | 110 g; water, 190 g | |
| Group | Group | Tremella | 12 mL | Soybean | 25° C., |
| B | B1 | aurantialba | | extruded powder, | 10 d |
| | | | | 120 g; water, 180 g | |
| | Group | Tremella | 21 mL | Soybean | 28° C., |
| | B2 | aurantialba | | extruded powder, | 20 d |
| | | | | 140 g; water, 160 g | |
| | Group | Tremella | 5.5 mL | Soybean | 30° C., |
| | B3 | aurantialba | | extruded powder, | 15 d |
| | | | | 110 g; water, 190 g | |
| Group | Group | Cordyceps | 18 mL | Soybean | 25° C., |
| C | C1 | sinensis | | extruded powder, | 10 d |
| | | | | 120 g; water, 180 g | |
| | Group | Cordyceps | 9 mL | Soybean | 28° C., |
| | C2 | sinensis | | extruded powder, | 20 d |
| | | | | 140 g; water, 160 g | |
| | Group | Cordyceps | 20 mL | Soybean | 30° C., |
| | C3 | sinensis | | extruded powder, | 15 d |
| | | | | 110 g; water, 190 g | |
| Group | Group | Hericium | 18 mL | Soybean | 25° C., |
| D | D1 | erinaceus | | extruded powder, | 15 d |
| | | | | 120 g; water, 180 g | |
| | Group | Hericium | 10 mL | Soybean | 28° C., |
| | D2 | erinaceus | | extruded powder, | 20 d |
| | | | | 140 g; water, 160 g | |
| | Group | Hericium | 15 mL | Soybean | 30° C., |
| | D3 | erinaceus | | extruded powder, | 10 d |
| | | | | 110 g; water, 190 g | |

TABLE 2
Content of bioactive substances in functional oil and fermented soybean meal

| | | | | Soybean meal (mg/g fermented soybean | | |
|---|---|---|---|---|---|---|
| | | Oil (mg/g functional oil) | | meal) | | |
| Group | | Triterpenoids | Flavonoids | Triterpenoids | Flavonoids | Polysaccharides |
| Blank group | | 22.413 | 0.038 | 8.416 | 0.130 | 6.805 |
| Group A | Group A1 | 60.270 | 0.047 | 10.259 | 1.599 | 40.486 |
| | Group A2 | 62.311 | 0.053 | 9.259 | 1.429 | 39.486 |
| | Group A3 | 65.663 | 0.062 | 10.457 | 1.468 | 40.107 |
| Group B | Group B1 | 52.834 | 0.053 | 13.511 | 1.596 | 43.838 |
| | Group B2 | 53.154 | 0.057 | 13.475 | 1.458 | 43.653 |
| | Group B3 | 56.372 | 0.064 | 13.626 | 1.493 | 43.372 |
| Group C | Group C1 | 48.879 | 0.058 | 13.651 | 1.020 | 35.133 |
| | Group C2 | 49.135 | 0.061 | 13.443 | 0.988 | 34.031 |
| | Group C3 | 51.652 | 0.067 | 13.592 | 1.004 | 33.203 |
| Group D | Group D1 | 27.913 | 0.247 | 14.028 | 0.291 | 59.983 |
| | Group D2 | 29.547 | 0.269 | 13.913 | 0.298 | 58.437 |
| | Group D3 | 33.215 | 0.302 | 14.225 | 0.304 | 60.651 |

TABLE 3
Type and content of fatty acid in functional oil (mg/g functional oil)

| | Blank | Group A | | | Group B | | |
|---|---|---|---|---|---|---|---|
| | group | Group A1 | Group A2 | Group A3 | Group B1 | Group B2 | Group B3 |
| C14:0 | 0.305 | 0.380 | 0.386 | 0.391 | 0.329 | 0.338 | 0.340 |
| C16:0 | 46.976 | 51.397 | 52.479 | 53.267 | 51.593 | 52.835 | 52.673 |
| C16:1 | 0.406 | 0.444 | 0.481 | 0.475 | 0.479 | 0.482 | 0.487 |
| C18:0 | 18.803 | 19.322 | 22.128 | 21.647 | 19.228 | 20.278 | 20.712 |
| C18:1 | 108.598 | 121.404 | 124.287 | 128.836 | 115.686 | 117.370 | 119.461 |
| C18:2 | 223.761 | 242.842 | 248.537 | 247.365 | 232.802 | 234.672 | 237.019 |
| C18:3 | 25.015 | 26.667 | 28.128 | 27.263 | 26.163 | 28.081 | 27.832 |
| C20:0 | 1.515 | 1.690 | 1.775 | 1.734 | 1.696 | 1.702 | 1.730 |
| C20:1 | 1.052 | 1.154 | 1.182 | 1.178 | 1.363 | 1.395 | 1.389 |
| C22:0 | 1.713 | 1.800 | 1.934 | 1.866 | 1.829 | 1.914 | 1.891 |
| SFA/UFA | 0.191 | 0.190 | 0.195 | 0.194 | 0.198 | 0.201 | 0.200 |

TABLE 4
Type and content of fatty acid in functional oil (mg/g functional oil)

| | Group C | | | Group D | | |
|---|---|---|---|---|---|---|
| | Group C1 | Group C2 | Group C3 | Group D1 | Group D2 | Group D3 |
| C14:0 | 0.343 | 0.351 | 0.357 | 0.298 | 0.316 | 0.308 |
| C16:0 | 52.948 | 53.768 | 53.186 | 45.207 | 46.269 | 46.781 |
| C16:1 | 0.493 | 0.511 | 0.507 | 0.414 | 0.423 | 0.468 |
| C18:0 | 19.637 | 21.070 | 21.379 | 20.927 | 22.078 | 21.145 |
| C18:1 | 117.199 | 120.603 | 121.003 | 88.721 | 89.647 | 91.436 |
| C18:2 | 240.180 | 242.878 | 242.161 | 226.951 | 229.340 | 230.471 |
| C18:3 | 27.452 | 28.182 | 28.089 | 33.386 | 34.895 | 34.240 |
| C20:0 | 1.736 | 1.790 | 1.801 | 1.460 | 1.503 | 1.521 |
| C20:1 | 1.148 | 1.163 | 1.172 | 0.740 | 0.782 | 0.793 |
| C22:0 | 1.884 | 1.939 | 1.957 | 1.538 | 1.679 | 1.782 |
| SFA/UFA | 0.198 | 0.200 | 0.200 | 0.198 | 0.202 | 0.200 |

Note:
SFA refers to saturated fatty acid;
SUFA refers to monounsaturated fatty acid;
PUFA refers to polyunsaturated fatty acid; and
UFA refers to unsaturated fatty acid.

It can be seen from Table 2 that the functional oil and soybean meal obtained by fermenting the extruded soybeans with the edible and medicinal fungi and further processing are rich in bioactive substances. The functional oil is rich in bioactive substances of triterpenes and flavonoids, and the soybean meal is rich in bioactive substances of triterpenes, flavonoids and polysaccharides. The bioactive substances endow the oil with the physiological functions of increasing host immunity, preventing vascular sclerosis, inhibiting tumors, resisting oxidation, and inhibiting free radicals, and also the functionality of the fermented soybean meal is improved and the feeding value is increased. It can be seen from Tables 3-4 that the content of different fatty acid in the functional oil does not change significantly, but the content of unsaturated fatty acid is rich, it indicates that the functional oil prepared by the disclosure has better health care effects.

### Example 2: Using Peanut Powder as Raw Material

Specific steps are as follows:

(1) The same as Example 1.

(2) The same as Example 1.

(3) Peanuts were put into a hot air circulating oven and dried at 50° C. until the water content reached 5-10%, then the peanuts were husked, and the husked peanuts were ground into fine peanut powder.

(4) The soybean extruded powder was replaced with the peanut powder. 300 g of a fermentation medium was added into a 2500 mL erlenmeyer flask (sterilized at 121° C. for 30 min), and the seed liquid was inoculated into the fermentation medium according to fermentation parameters in Table 1 for performing solid-state fermentation to obtain a fermented product.

Oil extraction was performed on the fermented product by analysis methods of functional oil and oil crop meal to obtain the functional oil and fermented peanut meal. The functional oil and fermented peanut meal were analyzed by the analysis methods of functional oil and oil crop meal, and it was found that both the functional oil and the fermented peanut meal are rich in bioactive substances. The functional oil is rich in bioactive substances of triterpenes and flavonoids, and the peanut meal is rich in bioactive substances of triterpenes, flavonoids and polysaccharides. The bioactive substances not only endow the oil with certain physiological functions, but also improve the quality of the peanut meal, thereby comprehensively improving the nutritional value of the oil and the peanut meal.

### Example 3: Using Rapeseed Powder as Raw Material

Specific steps are as follows:

(1) The same as Example 1.

(2) The same as Example 1.

(3) Rapeseeds were put into a hot air circulating oven and dried at 50° C. until the water content reached 5-10%, then the rapeseeds were husked, and the husked rapeseeds were ground into fine rapeseed powder.

(4) The soybean extruded powder was replaced with the rapeseed powder. 300 g of a fermentation medium was added into a 2500 mL erlenmeyer flask (sterilized at 121° C. for 30 min), and the seed liquid was inoculated into the fermentation medium according to fermentation parameters in Table 1 for performing solid-state fermentation to obtain a fermented product.

Oil extraction was performed on the fermented product by analysis methods of functional oil and oil crop meal to obtain the functional oil and fermented rapeseed meal. The functional oil and fermented rapeseed meal were analyzed by the analysis methods of functional oil and oil crop meal, and it was found that both the functional oil and the fermented rapeseed meal are rich in bioactive substances. The functional oil is rich in bioactive substances of triterpenes and flavonoids, and the rapeseed meal is rich in bioactive substances of triterpenes, flavonoids and polysaccharides. The bioactive substances not only endow the oil with certain physiological functions, but also improve the quality of the rapeseed meal, thereby comprehensively improving the nutritional value of the oil and the rapeseed meal.

Although the disclosure has been disclosed as above in preferred examples, the examples are not intended to limit the disclosure. Various changes and modifications can be made by anyone familiar with this technology without departing from the spirit and scope of the disclosure. Therefore, the protection scope of the disclosure should be defined by the claims.

1. A method of fermenting soybeans or other oil crops by edible and medicinal fungi, comprising the following steps: first, inoculating edible and medicinal fungi into a fermentation medium for performing solid-state fermentation to obtain a fermented product after solid-state fermentation, and then pressing the fermented product to obtain oil and oil crop meal;
wherein the edible and medicinal fungi are edible and medicinal fungi capable of producing bioactive substances;
the bioactive substances comprise one or more of active polysaccharides, flavonoids and triterpenoids;
components of the fermentation medium comprise oil crop powder and water;
the oil crop powder comprises one or more of soybean extruded powder, soybean powder, peanut powder, rapeseed powder, castor powder and sesame powder;
the components of the fermentation medium comprise the oil crop powder accounting for 40-50% of the total mass of the fermentation medium, and the water accounting for 50-60% of the total mass of the fermentation medium;
the edible and medicinal fungi comprise one or more of Ganoderma lucidum, Tremella aurantialba, Hericium erinaceus and Cordyceps sinensis;
conditions for the solid-state fermentation comprise the temperature of 25-30° C. and the time of 10-20 d; and
in inoculating the edible and medicinal fungi into the fermentation medium for performing solid-state fermentation, an edible and medicinal fungal seed liquid is inoculated into the fermentation medium for performing solid-state fermentation; the inoculation amount of the edible and medicinal fungal seed liquid in the fermentation medium is that the volume of an edible and medicinal fungal liquid accounts for 5-20% of the mass of the oil crop powder; and the edible and medicinal fungal seed liquid is obtained by inoculating an edible and medicinal fungus into a seed culture medium.

2. A method of fermenting soybeans or other oil crops by edible and medicinal fungi, comprising the following steps: first, inoculating edible and medicinal fungi into a fermentation medium for performing solid-state fermentation to obtain a fermented product after solid-state fermentation, and then pressing the fermented product to obtain oil and oil crop meal;
wherein the edible and medicinal fungi are edible and medicinal fungi capable of producing bioactive substances;
the bioactive substances comprise one or more of active polysaccharides, flavonoids and triterpenoids;
components of the fermentation medium comprise oil crop powder and water; and
the oil crop powder comprises one or more of soybean extruded powder, soybean powder, peanut powder, rapeseed powder, castor powder and sesame powder.

3. The method of claim 2, wherein the oil crop powder accounts for 40-50% of the total mass of the fermentation medium, and the water accounts for 50-60% of the total mass of the fermentation medium.

4. The method of claim 2, wherein the oil crop powder accounts for 40% of the total mass of the fermentation medium, and the water accounts for 60% of the total mass of the fermentation medium.

5. The method of claim 2, wherein the components of the fermentation medium comprise soybean extruded powder and water.

6. The method of claim 2, wherein the edible and medicinal fungi comprise one or more of Ganoderma lucidum, Tremella aurantialba, Hericium erinaceus and Cordyceps sinensis.

7. The method of claim 2, wherein the edible and medicinal fungus is Ganoderma lucidum.

8. The method of claim 2, wherein conditions for the solid-state fermentation comprise the temperature of 25-30° C. and the time of 10-20 days.

9. The method of claim 2, wherein the conditions for the solid-state fermentation comprise the temperature of 30° C. and the time of 10 days.

10. The method of claim 2, wherein in inoculating the edible and medicinal fungi into the fermentation medium for performing solid-state fermentation, an edible and medicinal fungal seed liquid is inoculated into the fermentation medium for performing solid-state fermentation; the inoculation amount of the edible and medicinal fungal seed liquid in the fermentation medium is that the volume of an edible and medicinal fungal liquid accounts for 5-20% of the mass of the oil crop powder; and the edible and medicinal fungal seed liquid is obtained by inoculating an edible and medicinal fungus into a seed culture medium.

11. Oil and oil crop meal prepared by the method of claim 2.

12. A product containing the oil and/or the oil crop meal of claim 11.

13. The product of claim 12, wherein the product is a feed, a food, a drug or a health care product.
